(19) United States
(12) Patent Application Publication
(10) Pub. No.: US 2015/0031697 A1
(43) Pub. Date: Jan. 29, 2015

(54) METHOD FOR PREVENTING ULTRAVIOLET RADIATION-INDUCED CUTANEOUS DAMAGE AND DEVELOPMENT OF SQUAMOUS CELL CARCINOMAS

(71) Applicant: WISCONSIN ALUMNI RESEARCH FOUNDATION, Madison, WI (US)

(72) Inventor: Ajit K. Verma, Madison, WI (US)

(21) Appl. No.: 14/341,570

(22) Filed: Jul. 25, 2014

Related U.S. Application Data
(60) Provisional application No. 61/858,986, filed on Jul. 26, 2013.
(60) Provisional application No. 62/007,705, filed on Jun. 4, 2014.

(51) Int. Cl. A61K 31/395; A61K 31/5377; A61K 31/496; A61K 31/519; A61K 31/416

(52) U.S. Cl. CPC A61K 31/395; A61K 31/519; A61K 31/416; A61K 31/496; A61K 31/5377
USPC 514/236.8; 514/183; 514/260.1; 514/406; 514/254.08

(57) ABSTRACT

Provided herein are methods of preventing skin cancer, where the methods comprise the step of administering an effective amount of at least one Hsp90 inhibitor to a subject at the risk of developing skin cancer, whereby the skin cancer is prevented in the subject.

### CROSS-REFERENCE TO RELATED APPLICATIONS

This application claims the benefit of U.S. Provisional Application No. 61/858,986, filed Jul. 26, 2013, and U.S. Provisional Application No. 62/007,705, filed Jun. 4, 2014, each of which is incorporated herein by reference as if set forth in its entirety.

### STATEMENT REGARDING FEDERALLY SPONSORED RESEARCH OR DEVELOPMENT

This invention was made with government support under CA035368 awarded by the National Institutes of Health. The government has certain rights in the invention.

### BACKGROUND OF THE INVENTION

Skin cancer is the second most common malignancy in the U.S., with 1.3 million new cases of non-melanoma skin cancer diagnosed each year (1). Malignant melanoma is a skin cancer in melanocytes (skin cells that make pigment). Basal cell carcinoma (BCC) is a skin cancer in the lower part of the epidermal (outer) skin layer. Squamous cell carcinoma (SCC) is skin cancer in squamous cells (flat cells at the skin surface). Skin cancer that forms in neuroendocrine cells (cells that release hormones in response to signals from the nervous system) is called neuroendocrine carcinoma of the skin. Most skin cancers form on skin tissues exposed to UV radiation or in people who have weakened immune systems. Squamous cell carcinoma (SCC) and basal cell carcinoma (BCC) are the most common human non-melanoma skin cancers (1,2). BCC is rarely life threatening because it is slow growing and is mostly localized. Unlike BCC, SCC invades other tissues (1), typically first to a regional lymph node, then to more distant sites such as the lung. SCC is mainly caused by cumulative exposure to UV radiation (UVR) over the course of a lifetime. Certain vulnerable populations, e.g., organ transplant recipients, have extraordinarily high rates of SCC (>50%) with markedly worse morbidity and mortality compared with the general public (3-6). To date, no accepted criteria define these SCC patients.

UV radiation (UVR) is radiation with a wavelength between about 100 nm and about 400 nm. UVR is generally divided into a number of different regions, including UVA (400-315 nm), UVB (315-280 nm), UVC (280-100 nm), NUV (400-300 nm), MUV (300-200 nm), FUV (200-122 nm), Hydrogen Lyman-α (122-121 nm), EUV (121-10 nm) and VUV (200-10 nm).

Hsp90 is a ubiquitous, highly conserved molecular chaperone protein that interacts with and stabilizes hundreds of client proteins, including oncogenic target proteins for cell transformation, proliferation, and survival (7,15,16). Hsp90 mediates maturation and stabilization of PKCε (7-12), a signal transduction pathway component important to UVR-induced SCC. Mammalian cells contain three types of Hsp90: cytosolic Hsp90, mitochondrial Trap-1, and glucose-regulated protein 94 (Grp94) of the endoplasmic reticulum. Each type of mammalian Hsp90, and their bacterial counterpart HtpG, hydrolyzes ATP and undergoes similar conformational changes. Unlike the other forms of Hsp90, cytosolic Hsp90 function requires a battery of co-chaperone proteins (17) that regulate the ATPase activity of Hsp90 or direct Hsp90 to interact with specific client proteins. Two human and mouse cytosolic Hsp90 isoforms, Hsp90α and Hsp90β, are encoded by gene Hsp90aa1 and Hsp90ab1, respectively. With 85.8% sequence identity and 93.4% similarity, the two isoforms are highly homologous. Whereas Hsp90β is more or less constitutively and ubiquitously expressed, the expression of Hsp90α is heat-inducible and more tissue-specific (18).

By inhibiting Hsp90, one can target a large number of downstream proteins and thereby attack the neoplastic process at several points. Geldanamycin, the first clinically-tested Hsp90 inhibitor, did not move forward in clinical trials due to liver toxicity. Second-generation derivatives, such as 17-allylamino-demethoxygeldanamycin (17-AAG) do not induce liver toxicity, have completed phase I, and are currently entering phase II clinical trials (23,26-30) in connection with leukemia or solid tumors, such as kidney, pancreatic, prostate, lung, and breast cancers. Hsp90 inhibitors have never been evaluated for prevention of SCC.

### SUMMARY OF THE INVENTION

The present invention relates to methods of using Hsp90 inhibitors to prevent skin cancer or skin damage caused by UV radiation. Specifically, the invention encompasses in a first aspect methods of preventing UV radiation-induced skin cancer comprising the step of administering to a subject at risk of developing skin cancer an effective amount of an Hsp90 inhibitor effective, wherein the skin cancer of the subject is prevented. The skin cancer that can be prevented by the disclosed method is basal cell carcinoma (BCC), squamous cell carcinoma (SCC) or malignant melanoma. Applicant envisions that the method would work best for preventing SCC.

In a second aspect, the invention encompasses methods of preventing UV radiation-induced skin damage comprising the step of administering an effective amount of at least one Hsp90 inhibitor to a subject at risk of UV radiation-induced skin damage, wherein the skin damage is prevented. The skin damage that can be prevented by the claimed method includes wrinkle, loss of elasticity, aging of skin, age spots, freckles, dryness, uneven skin tone, and mixtures thereof.

In either aspect of the present invention, Hsp90 inhibitors can be any one of 17AAG, NVP-AUY922, Elesclomol, Ganetespib, Alvespimycin, Geldanamycin, AT13387, KW-2478, SNX-2112, PF-04929113, NVP-BEP800, BIIB021, and derivatives or analogs thereof. Preferably, the Hsp90 inhibitor is 17AAG, NPV-BEP800 or SNX-2112. More preferably, the Hsp90 inhibitor is 17AAG or NPV-BEP800.

The effective amount for a human is the human equivalent dose converted from the effective amount for a non-human subject. One of the conversion methods is based on Body Surface Area (BSA) factors.

The suitable Hsp90 inhibitor can be administered to a subject through any methods known in the art. Preferably, Hsp90 inhibitor is administered to a subject through a topical route.

### BRIEF DESCRIPTION OF THE DRAWINGS

FIG. 1 presents data demonstrating that PKCε is a client protein of Hsp90β. (A) UVR increases the interaction of PKCε with Hsp90β. PKCε-overexpressing transgenic mice (line 224) were exposed once to UVR (4 kJ/m²), then sacrificed 1 hour or 2 hours after exposure. Epidermal protein extracts were prepared as described (37). The UVR source was Kodacel-filtered FS-40 sun lamps (approximately 60% UVB and 40% UVA). 100 micrograms of epidermal cell lysate protein was immunoprecipitated (IP) with the PKCε and immunoblotted (IB) with the Hsp90β antibody (Santa Cruz Biotechnologies, Santa Cruz, Calif.). (B) Co-localization of PKCε with Hsp90β in PKCε-overexpressing transgenic mouse skin harvested 24 hr after one UVR exposure (1.8 KJ/m2). (C), UVR-induced SCC in SKH-1 hairless mice and (D) human SCC. Briefly, paraffin-fixed tissue sections (˜5 μm) were treated with xylene and decreasing alcohol gradient. Because the epitope of interest was masked during fixation and could not bind to the antibody, we used antigen retrieval technology to reverse the masking Following antigen retrieval in citrate buffer, the sections were blocked in 10% goat and horse serum for 1 hour, and then incubated overnight at 4° C. with primary anti-PKCε antibody (anti-rabbit polyclonal), and anti-Hsp90β antibody (anti-mouse monoclonal). After the primary antibody, slides were washed with 1X Phosphate buffered saline (PBS) three times, incubated for 1 hour at room temperature with the respective conjugated secondary antibodies, anti-rabbit Rhodamine for PKCε, and anti-mouse FITC for Hsp90β, and covered with Dapi mounting media for nuclei staining Fluorescent pictures were captured in a Nuance™ fluorescence microscope through TRITC (red), FITC (green), and Dapi (blue) channels. Dapi/PKCε/Hsp90β is the merged image of the Dapi, FITC and TRITC channels. PKCε and Hsp90β were expressed in epidermis, sebaceous gland (SG), and hair follicle bulge region but not in differentiated epidermis.

FIG. 2 presents graphs demonstrating that 17AAG when applied topically to the skin is distributed both in epidermis and serum. SKH hairless mice (6-7 weeks old) were exposed to UVR (1.8 kJ/m²) three times weekly (Monday, Wednesday, and Friday). The mice in the vehicle group (n=3) received topical treatment of 200 μl vehicle (DMSO:acetone: 1:40 v/v) before and after UVR exposures. The mice in 17AAG group (n=3) received freshly prepared 500 nmol of 17AAG (DMSO:acetone: 1:40 v/v) before and after each UVR exposure. A group of mice (n=3) were also treated with 17AAG only. All mice were treated for 25 weeks and sacrificed at 1, 3, 6 and 24 hours following the last UVR exposure. (A) Blood samples were collected to analyze serum 17AAG by HPLC. (B and C) In a separate experiment, wild type FVB mice (6-7 weeks old) were treated once topically with 17AAG only or in conjunction with a single UVR (1.8 kJ/m2) exposure. Mice were sacrificed at 6 and 24 hours post-17AAG treatment. (B) Blood samples were collected for serum 17AAG analysis. (C) 17AAG level was also analyzed in the epidermal protein lysate. To prepare epidermal lysate, epidermis was scraped and homogenized in the lysis buffer. For 17AAG analysis, 50 μl of epidermal lysate was used. Shown are the 17AAG values normalized with total protein concentration. Each value is an average of duplicate samples. ND: Not detectable. Inset: Chemical structure of 17AAG.

FIG. 3 presents data demonstrating 17AAG's inhibition of (A) UVR-induced Hsp90β-PKCε interaction and PKCε expression levels, and (B) PKCε, Stat3, pStat3Tyr705, pStat3Ser727, Akt, and pAktser473 expression levels. PKCε-overexpressing transgenic mice (line 224) were exposed once to UVR (4 kJ/m2). 17AAG (500 nmol) or the vehicle was applied topically both before and after UVR exposure. Mice were sacrificed 24 hours post-UVR exposure. There were two mice per treatment point. Dorsal skin comprised excised, epidermal cell lysates prepared as described before (Aziz et al., 2007). Pooled epidermal cell lysate was analyzed for PKCε-Hsp90β interaction by immunoprecipitation/Western blot analyses (A). (B) 17AAG inhibits PKCε, Stat3, pStat3Tyr705, pStat3Ser727, Akt, pAktser473 expression levels. PKCε-overexpressing transgenic mice (line 224) were exposed once to UVR (2 kJ/m2). 17AAG or the vehicle was applied topically both before and after UVR exposure. Mice were sacrificed at 24 hours post-UVR exposure. There were three mice per treatment point. Dorsal skin comprised excised, epidermal cell lysates prepared as described before (Aziz et al., 2007). Pooled epidermal cell lysate was analyzed for PKCε expression level by western blot analysis (B). (C) Shown is the quantitation of the western blot (B).

FIG. 4 presents data demonstrating 17AAG's inhibition of UVR-induced hyperplasia and wrinkle formation in SKH1 mice accompanied decreased expression of matrix metalloproteinase (MMPs). Groups of SKH1 mice were exposed to UVR (2 kJ/m2) three times (Monday, Wednesday, Friday, and Monday) weekly for 6 and 12 weeks. Vehicle or 17AAG (500 nmol) was applied post each UVR exposure. Mice were sacrificed at 24 hours after the last UVR treatment. For morphological wrinkle examination, the pictures of dorsal side of live mice were taken using digital Canon camera. For histochemistry, skin specimens were fixed in 10% neutral buffered formalin for 24 hours and embedded in paraffin. To determine morphological and biochemical alteration in skin, the two layers of skin (epidermis and dermis) were isolated separately for analysis of the indicated protein by the western blot analysis. (A and B) Epidermal hyperplasia and epidermal thickness are illustrated, respectively, in vehicle and 17AAG treated SKH1 mice at 24 and 48 hours post-UVR. (C) Illustrates the gross morphological appearance of mice skin in vehicle+UVR treated and 17AAG+UVR treated groups at 6 weeks post UVR. (E) HE stained section of SKH-1 mice skin at 6 weeks post UVR. (D) Expression levels of MMP-2, MMP-9, Collagen IV, and Hsp70 at 6 and 12 weeks post UVR samples (pooled, n=3 each). AN: arbitrary number of the quantitation of the western blot. Epi: epidermis. Der: dermis, M1, M2 and M3 are sections from different mice.

FIG. 5 presents data demonstrating that 17AAG inhibits UVR-induced hyperplasia and the development of SCC in SKH hairless mice. The SKH-1 hairless mice (6-7 week old) were exposed to UVR. The UVR source was Kodacel-filtered FS-40 sun lamps (approximately 60% UVB and 40% UVA). Mice were exposed to UVR (1.8 kJ/m2) three times weekly (Monday, Wednesday and Friday). The mice in the vehicle group (n=18) received topical treatment of 200 μl vehicle (DMSO:acetone: 1:40 v/v) before and after each UVR exposures. The mice in the treatment group (n=13) received freshly prepared 500 nmol of 17AAG (DMSO:acetone: 1:40 v/v) before and after each UVR exposure. Carcinomas were recorded grossly as downward-invading lesions, which were confirmed histologically. The Kaplan-Meier (product-limit) “survival” curve across time is displayed (A). The number of UVR-induced SCC in 17AAG treated mice was statistically different at all weeks as compared to the vehicle treated mice (P=0.038). At the end of the experiment, mice were sacrificed. Representative photographs of at the end of the experiment (B). A part of the uninvolved dorsal skin was fixed for in 10% neutral-buffered formalin for histological examination. (C): H&E stained uninvolved dorsal skin sections from mice receiving indicated treatments. (D): A part of the uninvolved skin was also used to prepare total epidermal lysate to determine expression level of PKCε, Hsp90β, and Hsp70.

FIG. 6 presents data demonstrating that 17AAG inhibits UVR-induced development of SCC in PKCε overexpressing transgenic mice. The PKCε-overexpressing transgenic (line 224) (TG) and wildtype FVB/N mice (6-7 week old) were exposed to UVR. Mice were exposed to UVR (1.8 kJ/m2) three times weekly (Monday, Wednesday and Friday). The mice in the vehicle group received topical treatment of 200 μl vehicle (DMSO:acetone: 1:40 v/v) before and after UVR each exposure. The mice in treatment group received freshly prepared 500 nmol of 17AAG (DMSO:acetone: 1:40 v/v) before and after each UVR exposure. Carcinomas were recorded grossly as downward-invading lesions, which were confirmed histologically. The Kaplan-Meier (product-limit) “survival” curves across time (A and B). Representative photographs of indicated mice of at the end of the experiment (C and D). (E) 17AAG inhibits UVR-induced SCC even when applied post each UVR exposure. SKH-1 hairless mice (6-7 week old) were treated as described under (A) except 17AAG was applied post each UVR exposure. There were 11 mice in each group.

### DESCRIPTION OF THE INVENTION

A method for preventing skin cancer (SCC, BCC, or melanoma), includes the step of administering an amount of Hsp90 inhibitor to a subject at risk of developing skin cancer effective to prevent development of UV radiation-induced skin cancer. The inventor here discloses that Hsp90 inhibitors reduce the interaction between Hsp90 and PKCε, decrease PKCε expression level, and reduce SCC.

By “skin cancer”, the inventor means an unregulated cell growth that forms tumors in any part of skin tissues. For purposes of this invention, skin cancer can be any type of skin cancer caused by or associated with UVR exposure, or a combination of two or more such skin cancers. For example, in some specific embodiments, the skin cancer prevented is selected from the group consisting of BCC, SCC, and melanoma.

For the purpose of this invention, UVR exposure can be with ultraviolet light having a wavelength within any regions above or between, or in a combination of any two or more of these regions. For example, in some embodiments, the UV radiation is solar UV comprising UVC, UVB, and UVA. In some other embodiments, the UV radiation is a UV radiation generated by a lamp. Examples of the lamp emitting UVR include, but are not limited to carbon arc lamp, mercury vapor lamp, fluorescent lamp, tungsten filament lamp, photographic lamp and actinic-light lamp.

By practicing the methods of the invention on a subject, the inventor means on a animal, preferably on a mammal, and more preferably on a human. The subject can, but need not be a patient under the care of a physician or other health care professional. In some embodiments, the methods of the invention find use in experimental animals, in veterinary applications, and in the development of models for diseases in animals, including but not limited to rodents including mice, rats and dogs.

The subject can, but need not, have previously exhibited indicia of a skin cancer, or have previously been diagnosed with skin cancer, or can be an individual at high or low risk for developing skin cancer. “Risk of developing skin cancer” means an increased likelihood or probability of developing skin cancer, either from UVR exposure, or from other internal or external factors that predispose the subject to skin cancer upon exposure to UVR, or both, compared to a control subject who is less likely to develop skin cancer.

Specifically, the prevention is a process of precluding, delaying, averting, obviating, forestalling, stopping, hindering, reducing, and/or mitigating the onset or incidence of the progression of skin cancer. More specifically, the prevention is capable of invoking one or more of the following effects or clinical improvements:
(1) inhibition, to some extent, of skin tumor growth, including slowing down or complete growth arrest;
(2) reduction in the number of tumor cells;
(3) maintaining tumor size;
(4) reducing tumor size;
(5) inhibiting, including reducing, slowing down or completely preventing tumor cell infiltration into peripheral organs;
(6) inhibiting, including reducing, slowing down or completely preventing metastasis;
(7) enhancing anti-tumor immune response, which may result in maintaining tumor size, reducing tumor size, slowing the growth of a tumor, reducing, slowing or preventing invasion or reducing, slowing or preventing metastasis;
(8) relief, to some extent, of one or more symptoms associated with a skin cancer; and/or
(9) inhibiting, including reducing, slowing down or completely preventing tumor development and/or hyperplasia (i.e., the abnormal multiplication or increase in the number of new cells in a tissue or organ) in a subject at high risk for developing skin cancer (e.g., a transplant recipient or other immunocompromised individual).

The ability of an Hsp90 inhibitor to prevent UVR-induced skin cancer can be assessed in an animal model, including for example, mice, rats, rabbits, birds, cats, dogs, pigs, sheep, goats, deer, horses, cattle, and non-human primates.

The effective amount of an Hsp90 inhibitor to prevent UVR-induced skin cancer in a human can be determined in animal tests, and the scaling of the effective amount for human administration can be performed by any art-acceptable practices. For example, an amount can be initially measured to be effective in an animal model (e.g., achieve a desired effect of preventing UVR-induced skin cancer). The amount obtained from the animal model can be used in formulating an effective amount for humans by using conversion factors known in the art. The effective amount obtained in one animal model can also be converted for another animal by using suitable conversion factors.

Conversion of animal doses to human equivalent doses (HED) can be made based on Body Surface Area (BSA) factors. Each species has its own BSA factor. The BSA factors used by the US Food and Drug Administration (FDA) are listed in Table 1 below (31, 32):

TABLE 1
Conversion of Animal Doses to Human Equivalent Doses
Based on Body Surface Area

| | To Convert Animal | To Convert Animal Dose in mg/kg to | |
|---|---|---|---|
| | Dose in mg/kg to | HEDₐ in mg/kg, Either: | |
| | Dose in mg/m², | Divide | Multiply |
| Species | Multiply by kₘ | Animal Dose By | Animal Dose By |
| Human | 37 | — | — |
| Child | 25 | — | — |
| (20 kg)^{b} | | | |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel | 7 | 5.3 | 0.19 |
| Monkey | | | |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)^{0.33}.
^{b}This kₘ value is provided for reference only since healthy children will rarely be volunteers for phase 1 trials.
^{c}For example, cynomolgus, rhesus, and stumptail.

To perform the conversion, one can multiply the conversion factor listed above by an animal dose in mg/kg to obtain the dose for human dose equivalent. Specifically, the following formula is used to calculate a human dose equivalent:

For

$$Animal Dose ( \frac{mg}{kg} ) * \frac{Animal {K}_{m}}{Human {K}_{m}} = Human Equivalent Dose ( \frac{mg}{kg} )$$

example, if a topical dose of an Hsp90 inhibitor, such as 17-AAG (Tanespimycin), is 500 nmol for a 20 g mouse, the human equivalent dose is about 1.76 m/kg for a 20 kg child, about 1.19 mg/kg for a 60 kg adult, or about 1.02 mg/kg for a 90 kg adult. Table 2 describes the detailed steps of conversion:

TABLE 2
Conversion topical dose of 17-AAG in mice to a HED
Conversion of nmol to mg/kg

| Topical | Topical | 17 AAG | Topical | Topical | | Topical |
|---|---|---|---|---|---|---|
| Dose of 17 | Dose of | Molar | Dose of | Dose of | Mouse | Dose of |
| AAG in | 17 AAG | Weight | 17 AAG | 17 AAG | Weight | 17 AAG |
| mice (nmol) | in mice (mol) | (g/mol) | in mice (g) | in mice (mg) | (kg) | in mice (mg/kg) |
| 500 | 0.0000005 | 585.7 | 0.000293 | 0.29285 | 0.02 | 14.6425 |

Dose calculation

| | Dose | Human | | | | |
|---|---|---|---|---|---|---|
| | conversion | Equivalent | | Human Km | Human Km | Human Km |
| Human | (Animal Km/ | Dose of 17 | Mouse | for 20 kg | for 60 kg | for 90 kg |
| Weight (kg) | Human Km) | AAG (mg/kg) | Km | child | adult | adult |
| 20 kg Child | 0.1200 | 1.7571 | 3 | 25 | 37 | 43 |
| 60 kg Adult | 0.0811 | 1.1872 | | | | |
| 90 kg Adult | 0.0698 | 1.0216 | | | | |

To determine an effective amount of Hsp90 inhibitor for preventing UVR-induced skin cancer either in an animal or a human, one may, for example, evaluate the effects of a given inhibitor in a subject by incrementally increasing the dosage until the desired symptomatic relief level is achieved. A continuing or repeated dose regimen can also be used to achieve or maintain the desired result. Any other techniques known in the art can be used as well in determining the effective amount range. Of course, the specific effective amount will vary with such factors as the particular skin condition being treated, the physical condition of the subject, the type of animal being treated, the duration of the treatment, the nature of concurrent therapy (if any), and the specific formulations employed and the structure of a particular Hsp90 inhibitor or its derivatives or analogs.

During the prevention, an effective amount may be re-evaluated or adjusted. For example, the effective amount can be gradually decreased if the clinical condition of the skin tissues being treated has improved. It can be increased over time if the skin condition has not improved or becomes worse.

The administration of Hsp 90 inhibitors can be carried out by any methods or protocols known in the art. Also, the administration can vary depending on doses, dosage forms, formulations, compositions and/or administration devices involved.

In some embodiments, the inhibitor may be administered in forms for oral administration by means of tablets, troches, lozenges, sublingual absorption, and the like.

In some embodiments, the inhibitor may be administered by injection, including but not limited to subcutaneous administration, intradermal administration, subdermal administration, intramuscular administration, depot administration, intravenous administration or intra-arterial administration, and/or intra-cavitary administration (e.g., administration into the intrapleural or intraperitoneal space).

In some embodiments, the inhibitor may also be administered by implant, aerosol, nasal, buccal, vaginal, rectal, intratracheal, endoscopic, percutaneous administration or any other forms known in the art.

In a preferred embodiment, Hsp90 inhibitors are administered topically. In the broadest sense, any convenient topical administration that provides for the requisite penetration of Hsp90 inhibitors through the skin surface to the target area of the subject can be employed. Non-limiting examples of a suitable topical dosage form include:

(1) lotions (an emulsion, liquid dosage form, whereby this dosage form is generally for external application to the skin),

(2) lotion augmented (a lotion dosage form that enhances drug delivery, whereby augmentation does not refer to the strength of the drug in the dosage form),

(3) gels (a semisolid dosage form that contains a gelling agent to provide stiffness to a solution or a colloidal dispersion, whereby the gel may contain suspended particles),

(4) ointments (a semisolid dosage form, usually containing <20% water and volatiles and >50% hydrocarbons, waxes, or polyols as the vehicle, whereby this dosage form is generally for external application to the skin or mucous membranes) and ointment augmented (an ointment dosage form that enhances drug delivery, whereby augmentation does not refer to the strength of the drug in the dosage form),

(5) creams (an emulsion, semisolid dosage form, usually containing >20% water and volatiles and/or <50% hydrocarbons, waxes, or polyols as the vehicle, whereby this dosage form is generally for external application to the skin or mucous membranes) and cream augmented (a cream dosage form that enhances drug delivery, whereby augmentation does not refer to the strength of the drug in the dosage form),

(6) emulsion (a dosage form consisting of a two-phase system comprised of at least two immiscible liquids, one of which is dispersed as droplets, internal or dispersed phase, within the other liquid, external or continuous phase, generally stabilized with one or more emulsifying agents, whereby emulsion is used as a dosage form term unless a more specific term is applicable, e.g. cream, lotion, ointment),

(7) suspensions (a liquid dosage form that contains solid particles dispersed in a liquid vehicle) and suspension extended release (a liquid preparation consisting of solid particles dispersed throughout a liquid phase in which the particles are not soluble; the suspension has been formulated in a manner to allow at least a reduction in dosing frequency as compared to that drug presented as a conventional dosage form, e.g., as a solution or a prompt drug-releasing, conventional solid dosage form),

(8) pastes (A semisolid dosage form, containing a large proportion, 20-50%, of solids finely dispersed in a fatty vehicle, whereby this dosage form is generally for external application to the skin or mucous membranes),

(9) solutions (a clear, homogeneous liquid dosage form that contains one or more chemical substances dissolved in a solvent or mixture of mutually miscible solvents),

(10) powders,

(11) shampoos (a lotion dosage form which has a soap or detergent that is usually used to clean the hair and scalp; it is often used as a vehicle for dermatologic agents) and shampoo suspensions (a liquid soap or detergent containing one or more solid, insoluble substances dispersed in a liquid vehicle that is used to clean the hair and scalp and is often used as a vehicle for dermatologic agents),

(12) aerosol foams (i.e., a dosage form containing one or more active ingredients, surfactants, aqueous or nonaqueous liquids, and the propellants; if the propellant is in the internal discontinuous phase, i.e., of the oil-in-water type, a stable foam is discharged, and if the propellant is in the external continuous phase, i.e., of the water-in-oil type, a spray or a quick-breaking foam is discharged),

(13) sprays (a liquid minutely divided as by a jet of air or steam), including metered spray (a non-pressurized dosage form consisting of valves which allow the dispensing of a specified quantity of spray upon each activation) and suspension spray (a liquid preparation containing solid particles dispersed in a liquid vehicle and in the form of coarse droplets or as finely divided solids to be applied locally, most usually to the nasal-pharyngeal tract, or topically to the skin),

(14) jellies (a class of gels, which are semisolid systems that consist of suspensions made up of either small inorganic particles or large organic molecules interpenetrated by a liquid—in which the structural coherent matrix contains a high portion of liquid, usually water),

(15) films (a thin layer or coating), film extended release (a drug delivery system in the form of a film that releases the drug over an extended period in such a way as to maintain constant drug levels in the blood or target tissue), and film soluble (a thin layer or coating which is susceptible to being dissolved when in contact with a liquid),

(16) sponges (a porous, interlacing, absorbent material that contains a drug, whereby it is typically used for applying or introducing medication, or for cleansing, and whereby a sponge usually retains its shape),

(17) swabs (a small piece of relatively flat absorbent material that contains a drug, whereby a swab may also be attached to one end of a small stick, and whereby a swab is typically used for applying medication or for cleansing), and

(18) patches (a drug delivery system that often contains an adhesive backing that is usually applied to an external site on the body, whereby its ingredients either passively diffuse from, or are actively transported from, some portion of the patch, whereby depending upon the patch, the ingredients are either delivered to the outer surface of the body or into the body, and whereby a patch is sometimes synonymous with the terms ‘extended release film’ and ‘system’), patch extended release (a drug delivery system in the form of a patch that releases the drug in such a manner that a reduction in dosing frequency compared to that drug presented as a conventional dosage form, e.g., a solution or a prompt drug-releasing, conventional solid dosage form), patch extended release electronically controlled (a drug delivery system in the form of a patch which is controlled by an electric current that releases the drug in such a manner that a reduction in dosing frequency compared to that drug presented as a conventional dosage form, e.g., a solution or a prompt drug-releasing, conventional solid dosage form), and the like.

The various topical dosage forms may also be formulated as immediate release, controlled release, sustained release, or the like.

The topical dosage form composition may contain an active pharmaceutical ingredient and one or more inactive pharmaceutical ingredients such as excipients, colorants, pigments, additives, fillers, emollients, surfactants (e.g., anionic, cationic, amphoteric and nonionic), penetration enhancers (e.g., alcohols, fatty alcohols, fatty acids, fatty acid esters and polyols), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

The topical dosage form composition may also be prepared and administered in the presence of a pharmaceutically-acceptable carrier. A pharmaceutically-acceptable carrier can be a non-toxic material that does not interfere with the effectiveness of the Hsp90 inhibitor and that is compatible with the biological systems such as a tissue or organism. A pharmaceutically-acceptable carrier can also be a constituent such as penetration enhancer or other active ingredient. Examples of suitable pharmaceutically acceptable carriers include, but are not limited to diluents, fillers, salts, buffers, stabilizers, solubilizers and other materials which are well-known in the art.

The location of topical administration of the present invention can be any convenient topical site. For example, topical sites of interest include, but are not limited to, arms, legs, joints, face, neck, torso, etc. The topical administration can be employed to one or more distinct regions. In various embodiments, the topical administration of the inhibitor covers some or all of the UVR-exposed skin surface. For example, the inhibitors can cover 1%, 10%, 20%, 30%, 40%, 50%, 60%, 70%, 80%, 90%, 100% or more of the skin surface exposed to UVR.

The Hsp90 inhibitor can be applied once or a plurality of times over a given time period, where the inhibitor can be applied, for example, every minute, hourly, daily, weekly, biweekly, monthly, etc. The time during which the formulation is maintained at the application site depends on a variety of factors such as, but not limited to, the nature of the skin, the condition of the subject, and the sensitivities of the skin to UVR.

In addition to preventing skin cancer, the present invention may also be used to prevent damage caused by or associated with UVR exposure. The damage can be any kind of skin destruction such as wrinkle formation or loss of elasticity caused by UVR-induced disruption or degradation of connective tissue compounds such as collagen. Another example of skin destruction according to the present invention is premature aging of the skin, including UVR-induced lines, wrinkles, age spots, freckles, dryness and uneven skin tone.

Accordingly, by “reducing” or “preventing” skin damage, the inventor means a process which is capable of mitigating, or delaying skin destruction caused by or associated with UVR, so that the adverse physiological responses to UVR is reduced or prevented. It may also reduce, prevent, mitigate, or delay the addition of, or the augmentation of medically-unacceptable adverse effects caused by other internal or external factors that may otherwise damage skin upon exposure to UVR.

One skilled in the art can determine an effective amount of Hsp90 inhibitor according to a particularly desired result of preventing UVR-induced skin damage.

Hsp90 Inhibitors

The present invention involves using at least one Hsp90 inhibitor to prevent UVR-induced skin cancer or skin damage. By “Hsp90 inhibitor,” the inventor means any substance that inhibits the activity of the Hsp90 heat shock protein, including Hsp90α and Hsp90β. Preferably, the inhibitor is an Hsp90β inhibitor.

A “Hsp90 inhibitor” may also include its derivatives or analogs. By “derivatives” or “analogs” of a inhibitor, the inventor means any modifications of the inhibitor that have, or provide for, the same or similar biological function or activity as the inhibitor. Hsp90 inhibitors suited for use in methods of the invention can be naturally occurring or non-naturally occurring.

In some embodiments, the derivatives or analogs of an inhibitor include the modifications made on any one or more chemical groups of the inhibitor. In some embodiments, the derivatives or analogs of an inhibitor include the modifications made by changing the chemical structure of the inhibitor. In some other embodiments, the derivatives or analogs of an inhibitor may also include, but are not limited to, the tautomeric form, the stereoisomer, the polymorphs, the solvate, the pharmaceutically acceptable salt and ester/prodrug thereof

Non-limiting examples of commercially available Hsp90 inhibitors suited for use in methods of the invention are described below.

(1). 17-AAG (Tanespimycin)

17-AAG is a less toxic analogue of geldanamycin which binds to Hsp90 and alters its function. 17-AAG has a higher solubility in water or organic solvent, such as DMSO and ethanol, than Geldanamycin. 17-AAG displays a 100-fold higher affinity for Hsp90 derived from tumor cells compared to Hsp90 from normal cells. It binds into the ATP binding pocket in Hsp90 and induces the degradation of proteins that require this chaperone for conformational maturation. 17-AAG has the structure of:

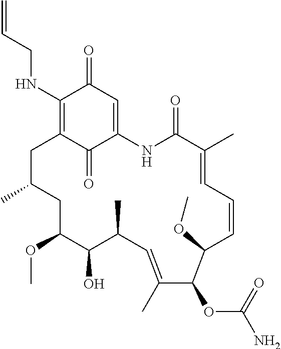 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

(2) 17-DMAG HCl (Alvespimycin).

17-DMAG HCl is a water soluble geldanamycin analog belonging to the class of benzoquinones. It binds to the N-terminal domain ATP binding site of Hsp90, inhibiting Hsp90 chaperone activity. 17-DMAG HCl demonstrates greater potency and water solubility than other geldanamycin analogs such as 17-AAG, while demonstrating anti-tumor activity and offering excellent oral bioavailability. It also displays ˜2 times potency against human Hsp90 than 17-AAG. 17-DMAG HCl has the structure of:

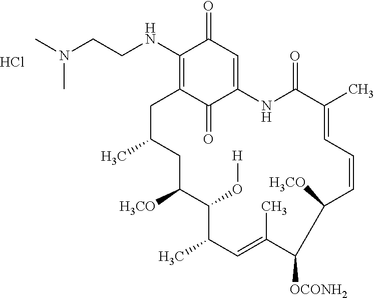

(3) NVP-AUY922 (AUY922).

NVP-AUY922 is a highly potent Hsp90 inhibitor for HSP90α and HSP90β having the structure of:

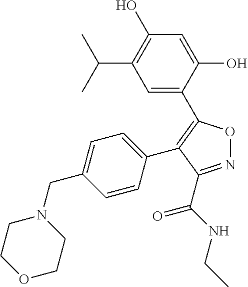 [SMILES: CCNC(=O)c1noc(-c2cc(C(C)C)c(O)cc2O)c1-c1ccc(CN2CCOCC2)cc1]

(4) Elesclomol.

Elesclomol is a small molecule that induces apoptosis via the mitochondrial apoptotic pathway in cancer cells by increasing oxidative stress, while having little or no effect on normal cells. It also significantly induces the expression of heat shock stress response genes and metallothionein genes. Elesclomol has the structure of:

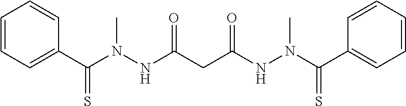 [SMILES: CN(NC(=O)CC(=O)NN(C)C(=S)c1ccccc1)C(=S)c1ccccc1]

(5) Ganetespib (STA-9090).

Ganetespib is a triazolone-containing Hsp90 inhibitor. It binds to the ATP-binding domain at the N-terminus of Hsp90 and acts as a potent Hsp90 inhibitor by degrading multiple oncogenic Hsp90 client proteins. Ganetespib has the structure of:

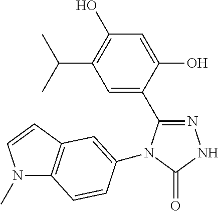 [SMILES: CC(C)c1cc(-c2n[nH]c(=O)n2-c2ccc3c(ccn3C)c2)c(O)cc1O]

(6) Geldanamycin

Geldanamycin is a benzoquinone ansamycin antibiotic that binds to Hsp90 and induces the degradation of proteins that are mutated in tumor cells preferentially over their normal cellular counterparts. Despite its potent antitumor potential, geldanamycin presents several major drawbacks as a drug candidate (namely, hepatotoxicity) that have led to the development of geldanamycin analogues, in particular analogues containing a derivatization at the 17 position, such as 17-AAG and 17-DMAG. Geldanamycin has the structure of:

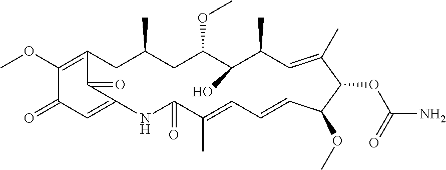 [SMILES: COC1=C2C[C@@H](C)C[C@H](OC)[C@H](O)[C@@H](C)/C=C(\C)[C@H](OC(N)=O)[C@@H](OC)/C=C/C=C(\C)C(=O)NC(=CC1=O)C2=O]

(7) AT13387

AT13387 is a selective potent small molecule Hsp90 inhibitor having the structure of:

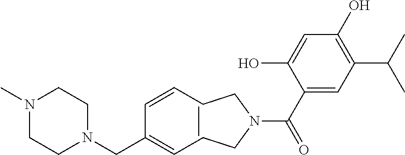 [SMILES: CC(C)c1cc(C(=O)N2CC/C=C\C(CN3CCN(C)CC3)=C/CC2)c(O)cc1O]

(8) KW-2478

KW-2478 is a non-ansamycin potent Hsp90 inhibitor. It is also a non-purine analogue antagonist for Hsp90. KW-2478 displayed a significant inhibition of tumor growth by inducing the degradation of client proteins to Hsp90 in tumor cells. KW-2478 has the structure of:

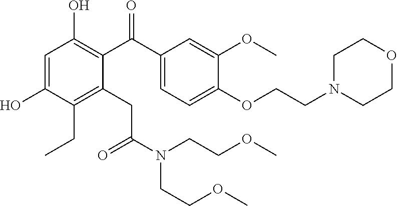 [SMILES: CCc1c(O)cc(O)c(C(=O)c2ccc(OCCN3CCOCC3)c(OC)c2)c1CC(=O)N(CCOC)CCOC]

(9) SNX-2112

SNX-2112 is a potent synthetic Hsp90 inhibitor having the structure of:

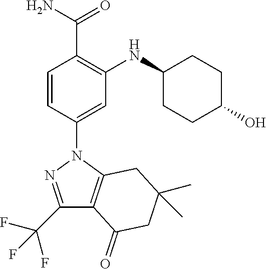 [SMILES: CC1(C)CC(=O)c2c(C(F)(F)F)nn(-c3ccc(C(N)=O)c(N[C@H]4CC[C@H](O)CC4)c3)c2C1]

(10) PF-04929113 (SNX-5422)

PF-04929113 is a potent and selective Hsp90 inhibitor with potential antineoplastic activity having the structure of:

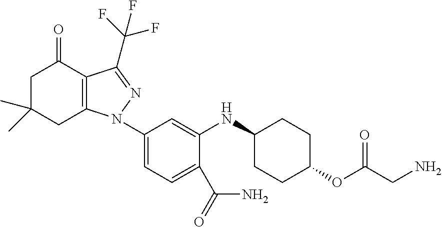 [SMILES: CC1(C)CC(=O)c2c(C(F)(F)F)nn(-c3ccc(C(N)=O)c(N[C@H]4CC[C@H](OC(=O)CN)CC4)c3)c2C1]

(11) NVP-BEP800

NVP-BEP800 is a fully synthetic, orally bioavailable Hsp90 inhibitor that binds to the N-terminal ATP-binding pocket of Hsp90 and has the structure of:

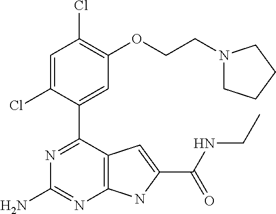 [SMILES: CCNC(=O)c1cc2c(-c3cc(OCCN4CCCC4)c(Cl)cc3Cl)nc(N)nc2[nH]1]

(12) BIIB021.

BIIB021, formerly CNF2024, is a fully synthetic Hsp90 inhibitor. It binds competitively with geldanamycin in the ATP-binding pocket of Hsp90. BIIB021 has the structure of:

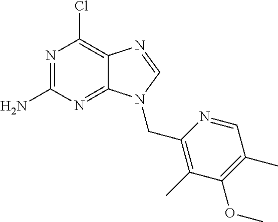 [SMILES: COc1c(C)cnc(Cn2cnc3c(Cl)nc(N)nc32)c1C]

Hsp90 inhibitors can be any one of, or mixtures of the compounds listed above or the derivatives or analogs thereof. Preferred Hsp90 inhibitors include 17-AAG, NPV-BEP800 and SNX-2112.

It should be understood that the present invention has been described above with respect to its preferred embodiments. Other forms of this concept are also intended to be within the scope of the claims.

### EXAMPLES

Examples described herein demonstrate a preliminary study of whether Hsp90 inhibitors prevent the development of UVR-induced skin cancer. Briefly, the inventor evaluated the effects of topically applied 17-AAG on UVR-induced development of cutaneous SCC in mice. In experiments with three separate mouse lines (wild-type FVB, PKCε overexpressing transgenic FVB and SKH-1 hairless mice), topical application of 17-AAG increased the latency and decreased both the incidence and multiplicity of UVR-induced cutaneous SCC. Also, 17-AAG treatment suppressed UVR-induced Hsp90β-PKCε, decreased PKCε expression level and inhibited UVR-induced epidermal carcinomas.

It is to be understood, however, that these examples are provided by way of illustration and nothing herein should be taken as a limitation upon the overall scope of the invention.

### Example 1

### Materials and Methods

Chemicals, Antibodies, and Assay Kits:

The antibodies to PKCε, Hsp90β, Hsp70, MMP-2, MMP-9, β-actin were from Santa Cruz Biotechnologies (Santa Cruz, Calif.); Stat3, pStat3Ser727, pStat3Tyr705, Akt, and pAktSer473 from Cell Signaling; collagen IV from Abeam; Anti-mouse, anti-goat, and anti-rabbit secondary antibodies were purchased from Thermo Scientific (Rockford, Ill.). 17-AAG was purchased from LC Laboratories (Woburn, Mass.). 17-AAG has >99% purity determined by HPLC analysis.

Mice and UVR Treatment:

SKH1 hairless mice were purchased from Charles River Laboratories (Wilmington, Mass.). PKCε transgenic mice were generated as described previously (Reddig et al., 2000). The UVR source was Kodacel-filtered FS-40 sunlamps (approximately 60% UV-B and 40% UV-A). Mice were exposed to UVR from a bank of six Kodacel-filtered sunlamps. UVR dose was routinely measured using UVX-radiometer.

Western Blot Analysis:

Mouse skin was excised and scraped to remove subcutaneous fat. The epidermis was scraped off on an ice-cold glass plate, homogenized in lysis buffer [50 mmol/L HEPES, 150 mmol/L NaCl, 10% glycerol, 1% Triton X-100, 1.5 mmol/L MgCl₂, 10 μg/m aprotinin, 10 μg/m leupeptin, 1 mmol/L phenylmethylsulfonyl fluoride (PMSF), 200 μmol/L Na3VO4, 200 μmol/L NaF, and 1 mmol/L EGTA (final pH 7.5)]. The homogenate was centrifuged at 14,000 g for 30 min at 4° C. Epidermal cell lysate proteins were fractionated on 10% Criterion™ pre-cast SDS-polyacrylamide gels (Bio-Rad Laboratories, Hercules, Calif.). The protein was transferred to 0.45 μm Hybond-P polyvinylidene difluoride (PVDF) transfer membrane (Amersham Life Sciences, Piscataway, N.J.). The membrane was then incubated with the indicated antibody followed by a horseradish peroxidase secondary antibody (Thermo Scientific), and the detection signal was developed with Amersham's enhanced chemiluminescence reagent and using FOTO/Analyst Luminary Work Station (Fotodyne Inc.). The Western blots were quantitated by densitometric analysis using Totallab® Nonlinear Dynamic Image analysis software (Nonlinear USA, Inc., Durham, N.C.).

Immunoprecipitation Protocol:

Epidermal lysates were prepared as for Western blot analysis. 100 μg of epidermal lysate was incubated with 10 μg of the indicated antibody. The total volume of the lysate/antibody mixture was adjusted to 1,000 μL with lysis buffer to allow for appropriate mixing and rotated at 4° C. overnight. Lysate/antibody mixture was then mixed with 50 μL of protein agarose A/G (sc-2003 Santa Cruz Biotechnology, Santa Cruz, Calif.) for 6 h. Lysate/antibody/protein A/G agarose mixture was then centrifuged at 8,000 g for 10 min to sediment the protein A/G agarose. Pellet was washed with 0.1% tween in PBS and then sedimented at 8,000 g for 10 min three times to wash any non-specific binding from the pellet. After three washes the immunoprecipite was then boiled for 5 min in 20 μL Protein Loading Buffer Blue (Cat # EC-886, National Diagnostics, Atlanta, Ga.). Immunoprecipitates were then treated as in Western Blot analysis above.

HPLC Analysis of 17-AAG in Serum and Mouse Epidermis:

Dorsal areas of the mice (6-7 week old) were shaved and depilated one day before the treatment. 17AAG stock (100 mM) was prepared in DMSO and freshly reconstituted in acetone to a desired concentration at the time of treatment. 17AAG or vehicle (200 μl) was applied topically to skin either alone or in conjunction with UVR exposures. Blood samples were collected to detect 17AAG in serum. To prepare epidermal lysate, epidermis was removed and homogenized with the lysis buffer. 17-AAG levels in the serum and mouse epidermis were analyzed by HPLC (Shin, et al., 2012).

Results

PKCε is a Client Protein of Hsp90β.

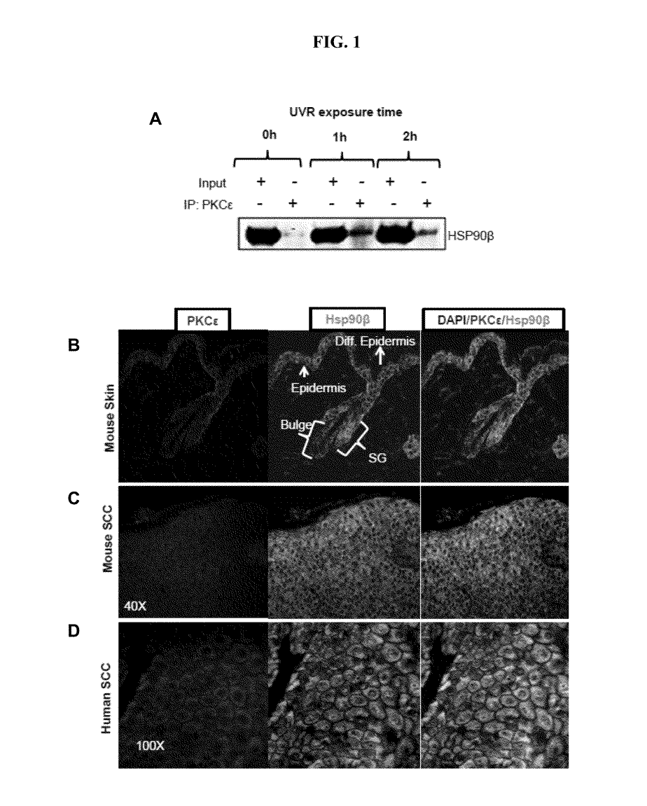

As shown in FIG. 1, PKCε transgenic mice were exposed once to UVR (4 kJ/m2). PKCε-Hsp90 interaction was analyzed by co-immunoprecipitation and Western blotting, at 1 and 2 hr post UVR treatment. As shown FIG. 1A, PKCε co-immunoprecipitates with Hsp90β. PKCε-Hsp90β interaction was enhanced as early as 1 hr post UVR treatment. To confirm the association of PKCε with Hsp90β, we determined the colocalization of PKCε and Hsp90β by double immunofluorescence staining 5-μm-thick sections from paraffin-fixed samples from the mice were used. PKCε and Hsp90β localization is indicated by the presence of red and green fluorescence, respectively. The yellow fluorescence indicates colocalization and association of PKCε with Hsp90β. The co-localization of PKCε with Hsp90 was observed in mouse epidermis (FIG. 1B), UVR-induced SCC in SKH-1 hairless mice (FIG. 1C) and human SCC (FIG. 1D). The expression of PKCε and Hsp90β was seen in epidermis, sebaceous gland, and bulge region of hair follicle. However, the expression of PKCε and Hsp90β was lost in differentiated epidermis keratinocytes.

Topical 17AAG Inhibited UVR-Induced Induction of SCC that Accompanied Decrease in: 1) Hyperplasia, and 3) Hsp90β-PKCε Interaction, Hsp90β, PKCε, Stat3, pStat3Ser727, pStat3Tyr705, Akt, pAktSer473 and Matrix Metalloproteinase (MMPs) Expression Levels.

17AAG is a competitive inhibitor of ATP binding and inhibits Hsp90 ATPase activity (Stebbins, et al., 1997). Inhibition of Hsp90 ATPase activity by 17AAG results in disruption of heteroprotein complexes and blocking of the refolding, conformational maturation and stability of transforming proteins including PKCε (Gould, et al., 2009). This led to us to hypothesize that disruption of UVR-induced interaction of Hsp90β oncogenic client proteins by Hsp90 inhibitor 17AAG will result in the prevention of UVR-induced SCC. To test this hypothesis, 17AAG was applied topically to skin in conjunction with each UVR exposure. Noteworthy observations were as follows:

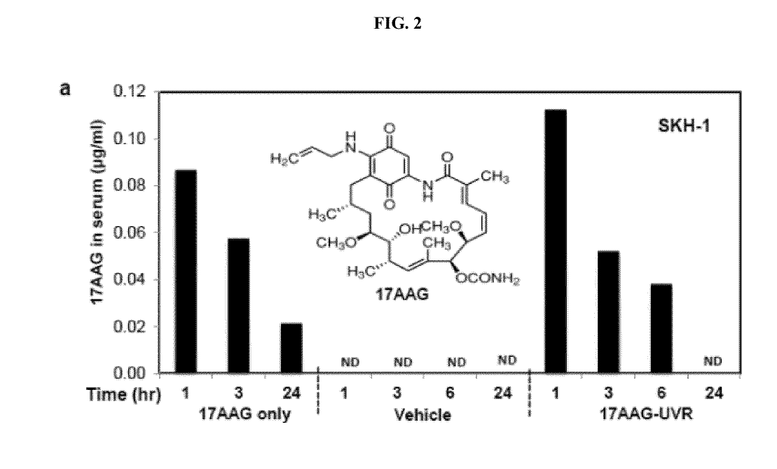

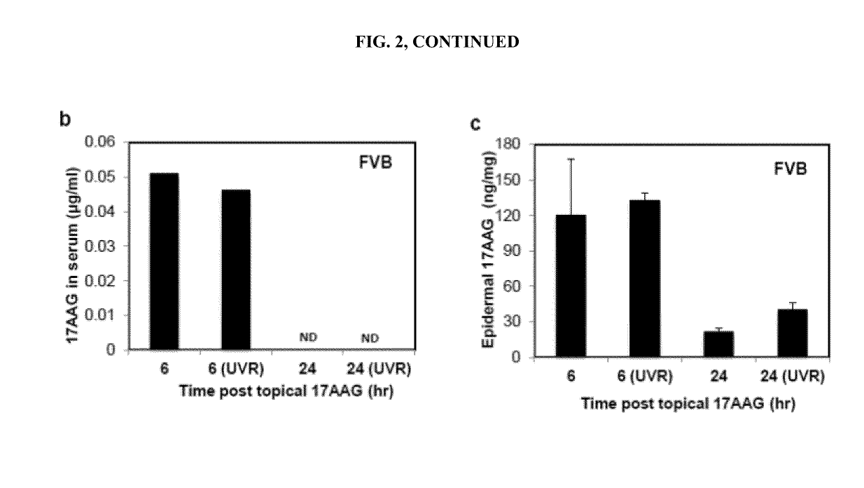

1. Topically applied 17AAG is distributed both in epidermis and serum. In all the foregoing experiments, stock solution of 17AAG (100 mM) was prepared in DMSO and then diluted with acetone (DMSO:acetone: 1:40 v/v) to obtain desired dose for topical application to skin. As a prelude to evaluate the biochemical and biological effects of topical 17AAG on UVR exposed skin, we determined its distribution in skin and serum. In these experiments (FIG. 2), 17AAG solution (DMSO:acetone: 1:40 v/v) was applied alone or in conjunction with either acute or chronic UVR exposures. A time course of topically applied 17AAG on epidermal and serum levels is shown in FIG. 2A. In this experiment (FIG. 2), SKH-1 hairless mice received topical 17AAG treatment in conjunction with each of three weekly UVR exposures for 25 weeks. Mice were sacrificed for serum 17AAG analysis at 1, 3, 6, and 24 hr after last UVR exposure. Maximum serum 17AAG level was detected as early as 1 hr post topical 17AAG application (FIG. 2A). In a separate acute UVR exposure experiment with FVB mice, the level of 17AAG was analyzed in both serum and epidermis at 6 and 24 hours post 17AAG application (FIG. 2B). Both serum (FIG. 2B) and epidermal 17AAG (FIG. 2C) level was detectable at 6 hr after 17AAG treatment. In both experiments with SKH-1 and FVB mice, serum (FIGS. 2A-B) and epidermal 17AAG (FIG. 2C) levels were lowest at 24 hours post-17AAG treatment. UVR treatment does not appear to significantly affect either the serum or epidermal 17AAG level (FIG. 2).

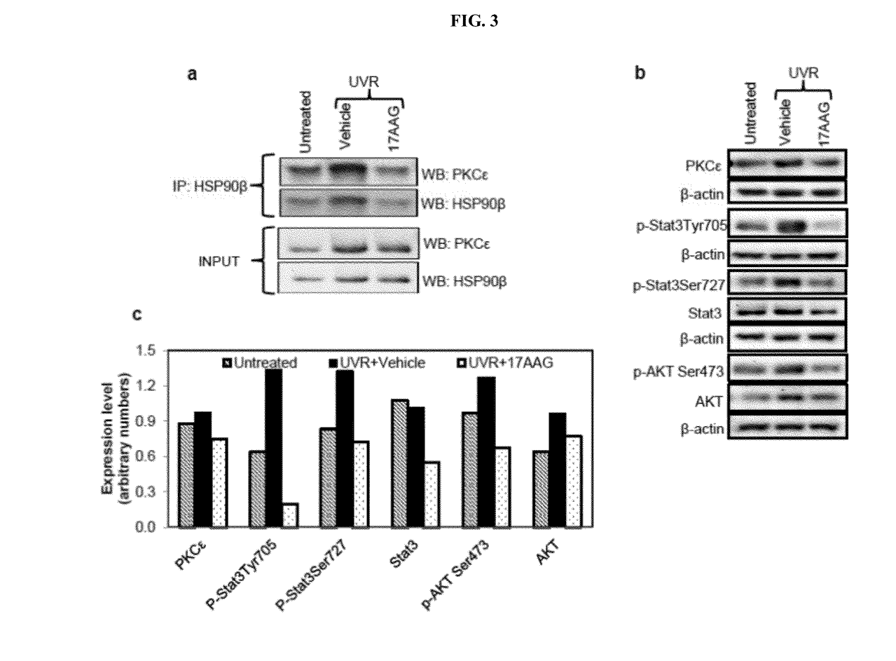

2. Topical 17AAG treatment suppressed Hsp90β-PKCε interaction, Hsp90β, Stat3, pStat3Ser727, pStat3Tyr705, Akt, pAktSer473 expression levels. In this experiment (FIG. 3), PKCε-overexpressing FVB transgenic mice (line 224) were exposed once to UVR. 17AAG (500 nmol) or the vehicle was applied topically both before and after UVR exposure. Mice were sacrificed 24 hours post-UVR exposure. UVR exposure enhanced Hsp90β-PKCε interaction and 17AAG treatment in conjunction with UVR exposure suppressed PKCε-Hsp90β interaction (FIG. 3A), but did not appreciably affect PKCε expression level (FIG. 3A, input). Also, 17AAG treatment in conjunction with UVR inhibited Stat3, pStat3Ser727, pStat3Tyr705 and pAktser473 expression levels (FIGS. 3B-C).

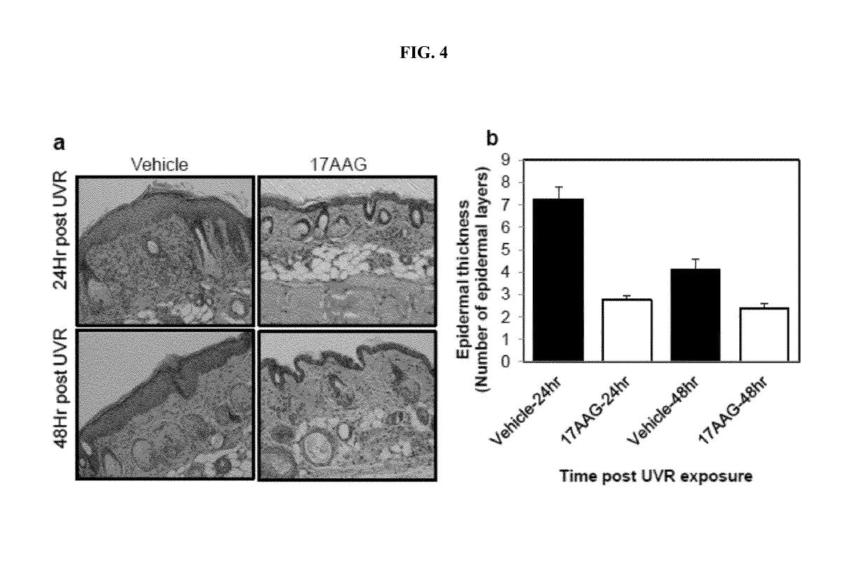

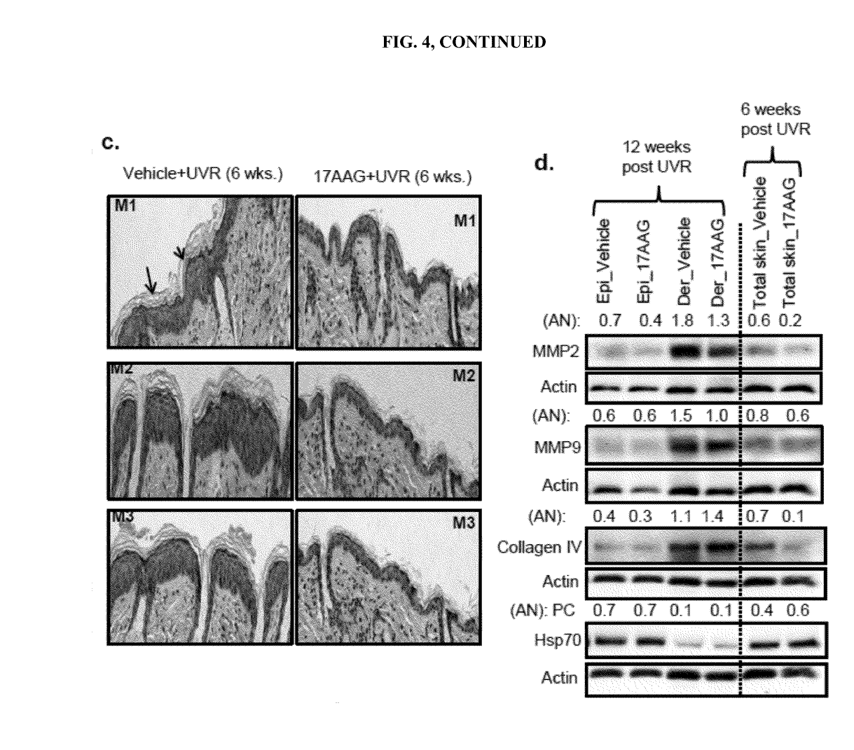

3. 17AAG-caused inhibition of UVR-induced hyperplasia in SKH1 mice accompanied decreased expression of matrix metalloproteinase (MMPs). In this experiment, groups of SKH1 hairless mice were exposed either four times to UVR (4 kJ/m², Monday, Wednesday, Friday, and Monday) or for 6 and 12 weeks (2 kJ/m², thrice weekly, Monday, Wednesday, Friday). 17AAG (500 nmol) or the vehicle was applied topically after each UVR exposure. The mice were sacrificed at 24 hr and 48 hr after the fourth UVR exposure. The, dorsal skin was removed and fixed in 10% formalin for the analyses of epidermal hyperplasia. 17AAG treatment inhibited UVR-induced hyperplasia as indicated by significantly (p<0.01) inhibition of epidermal thickness (FIGS. 4A-B). 17AAG-caused inhibition of UVR-induced hyperplasia (FIG. 4C) accompanied decrease in expression level of MMP-2, MMP-9, collagen IV and increase in the expression of Hsp70 (FIG. 4D). Also, topical 17AAG inhibited UVR-induced proliferative marker PCNA.

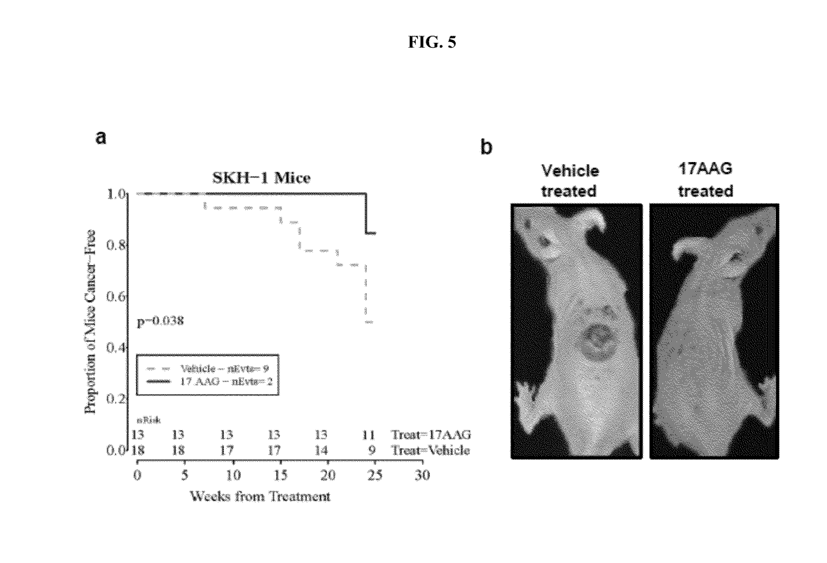

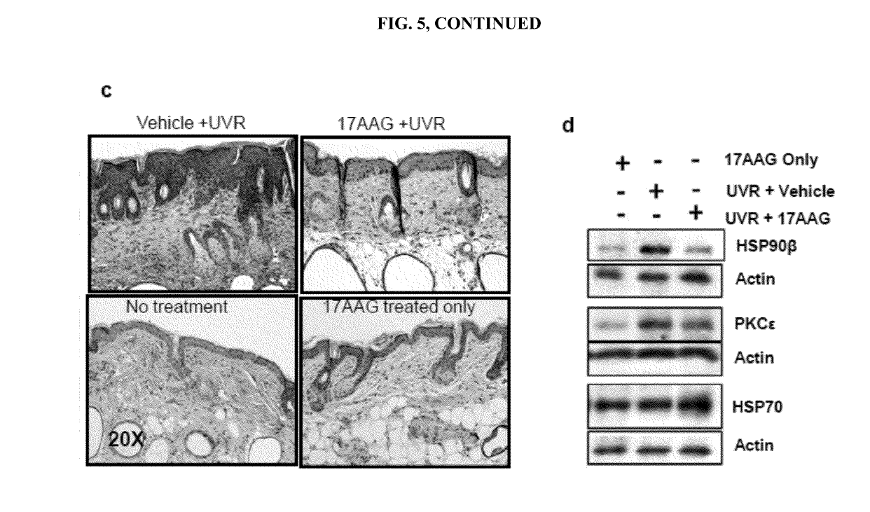

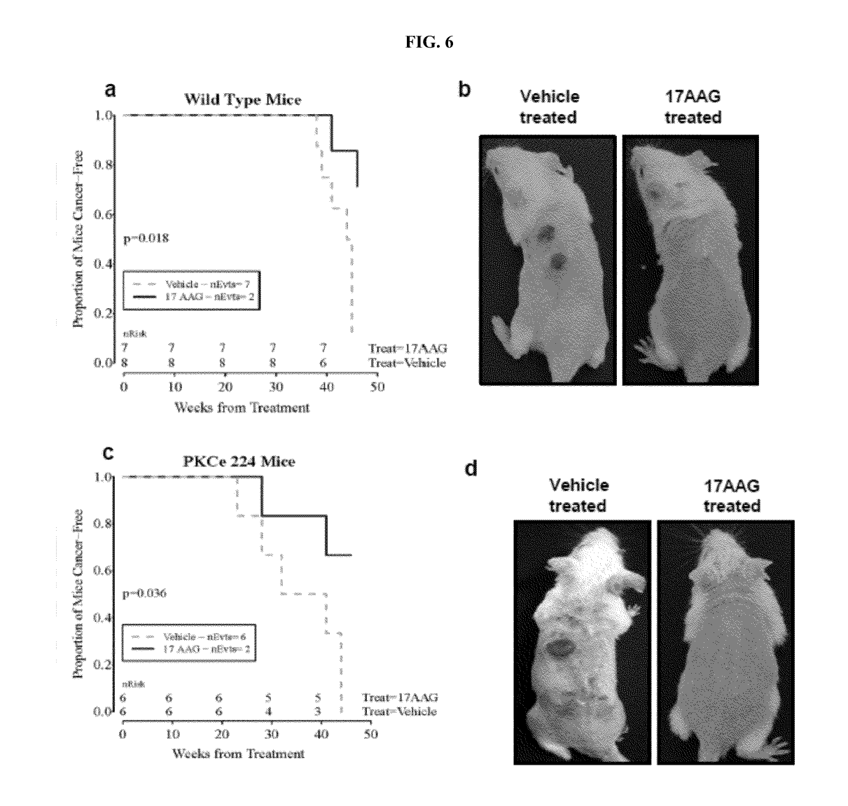

4. Topical 17AAG treatment inhibited UVR-induced development of SCC. Initial tumor induction experiment was performed with SKH-1 hairless mice. The Kaplan-Meier (product-limit) “survival” curve across time is displayed (FIG. 5A). Topical 17AAG increased SCC latency by 17 weeks and significantly (p=0.038) inhibited SCC incidence in SKH hairless mice (FIG. 5A). 17AAG-caused inhibition of UVR-induced SCC (FIG. 5A-B) accompanied inhibition of UVR-induced hyperplasia (FIG. 5C), Hsp90β and PKCε expression levels (FIG. 5D) and an increase in Hsp70 level (FIG. 5D).

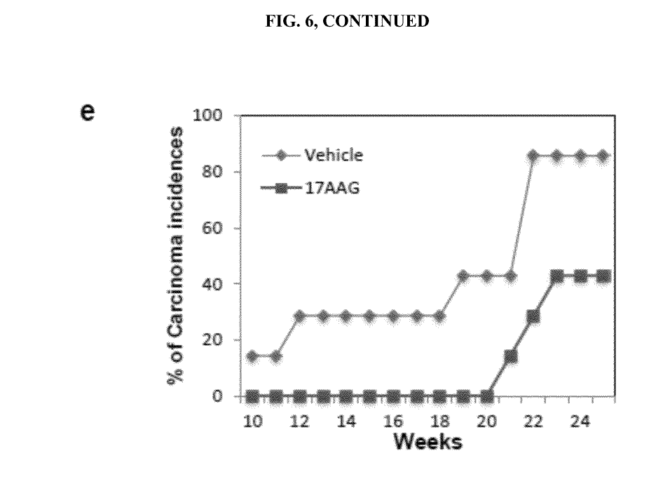

The effects of topical 17AAG on UVR-induced development of SCC, was further investigated using PKCε overexpressing transgenic FVB/N mice. We have previously reported that epidermal PKCε level dictates the susceptibility of transgenic mice to the development of SCC elicited by either the repeated exposure to UVR or using the DMBA-TPA tumor promotion protocol (Wheeler et al., 2004; Sand et al., 2010; Verma et al., 2006; Aziz, et al., 2007; Reddig, et al., 2000; Jansen, et al., 2001a; Jansen, et al., 2001b; Wheeler, et al., 2003). As compared to wildtype littermates, PKCε over-expressing transgenic mice exhibit decreases in tumor latency and increases in SCC multiplicity (Wheeler et al., 2004; Sand et al., 2010). Again, topical application of Hsp90 inhibitor 17AAG in conjunction with UVR exposures significantly inhibited the development of SCC in both PKCε overexpressing transgenic FVB/N mice (p=0.036) and wild-type littermates (p=0.018) (FIG. 6).

In all the above described tumor induction experiments (FIGS. 5 and 6), 17AAG was applied before and after each UVR exposure. We further explored the possibility that the inhibitory effects of 17AAG on UVR-induced development of SCC, is not attributable to a sunscreen property. In this experiment, 17AAG was applied post each UVR exposure (FIG. 6E). Topical 17AAG increased SCC latency by 20 weeks and inhibited more that 50% SCC incidence (FIG. 6E).

### DISCUSSION

Cutaneous SCC is mainly caused by cumulative UVR exposure over the course of a lifetime. UVR is a complete carcinogen, which both initiates and promotes carcinogenesis. UVR initiates photocarcinogenesis by directly damaging DNA (5-7). The tumor promotion component of UVR carcinogenesis, which involves clonal expansion of the initiated cells, is probably mediated by aberrant expression of genes altered during tumor initiation. UVR has been reported to alter the expression of genes regulating inflammation, cell proliferation and migration, cell differentiation, angiogenesis and metastasis. Specific examples include up-regulation of the expression of p21 (WAFT/C1P1; ref. 10), p53 (8), AP-1 activation (11), ornithine decarboxylase (ODC; ref. 12), COX2 (13), TNFα, and a wide variety of cytokines and growth factors (14), peroxixome proliferator-activated (PPAR) β/δ, oncogene Src ( ), Matrix mettaloproteinase (MMP), Stat3, and PKCε. Many of these molecular regulators of UVR carcinogenesis are acutely dependent on Hsp90 for maturity, stability and activity. These evidences prompted us to test the hypothesis that treatment of Hsp90 inhibitor in conjunction with UVR exposures will prevent development of SCC. We now present for the first time that topical Hsp90 inhibitor 17AAG applied in conjunction with each UVR exposure increased the latency and decreased both the incidence and multiplicity of UVR-induced SCC.

Topically applied 17AAG (500 nmol in acetone/DMSO vehicle (DMSO:acetone: 1:40 v/v) to skin is rapidly distributed both in epidermis and serum (FIG. 2). Since, both epidermis and serum level of 17AAG declined at 24 hours post-treatment, topically applied 17AAG also has short half-life (FIG. 2). The pharmacokinetics of 17AAG in patients and mice have been reported (Saif et al., 2013; Weigel et al., 2007; Bagatell et al., 2007; Goetz et al., 2005, Egorin et al., 2001). In these reports 17AAG was administered intravenous or orally. Half-life of 17AAG varied from 3-4 hours. The pharmacokinetics of systemically administered 17AAG have also been reported in mice (Egorin et al., 2001). 17AAG has excellent bioavailability when give intraperitoneally but only modest bioavailability when given orally (Saif et al., 2013; Weigel et al., 2007; Bagatell et al., 2007; Goetz et al., 2005, Egorin et al., 2001).

About 200 oncogenic proteins have been identified as clients of Hsp90 (3,20,21). However, UVR-induced mouse epidermal protein clients of Hsp90β remain to be identified. Results from reciprocal co-immunoprecipitation experiments (FIGS. 1 and 3) indicate that PKCε is a client protein of Hsp90β. UVR treatment increases the interaction of PKCε with Hsp90β (FIG. 1). UVR exposure of mouse skin results in increased expression of PKCε, possibly due to its increased synthesis (data not shown). Newly synthesized PKC undergoes well-ordered sequential phosphorylation for activation and Hsp90 binds newly synthesized PKCε, a required step in its maturation and enzyme stability (Gould et al., 2009). Obvious UVR treatment-caused interaction of PKCε with Hsp90β appears to be the result of increased expression of newly synthesized PKCε. Topical application of 17AAG to skin inhibited UVR-induced Hsp90β-PKCε interaction (FIG. 2). However, 17AAG did not appreciably affect PKCε protein level.

Epidermal hyperplasia due to inflammation, skin damage and alteration in extra-cellular matrix (ECM) (such as damage to collagen and elastic fibers, low collagen expression, and disruption of epidermal membrane) has an important role in UV-induced wrinkle formation (Talwar et al, 1995; Imokawa, 2009). UVR exposure induces wrinkle formation in dorsal skin of hairless SKH1 mice (Schwartz, 1988). UV exposure has been shown to increase the expression level of matrix metaloproteinases (MMP-1, 2, 3, and 9) both in mouse and human skin (Inomata et al., 2003; Rabe et al., 2006). Inhibition of MMP-2 and MMP-9 has been shown to protect from UVB-induced wrinkling and photoaging (Matsuda et al., 2012, Fischer et al., 1996, Jung et al., 2010). In Consistent with these reports, we also observed a decrease in the protein expression of MMP-2, MMP-9, and increase in Hsp70 in 17AAG treated SKH1 mice compared to their respective controls at 6 weeks post UVR exposure. These results may be due to the inhibitory effects of 17AAG on Hsp90, which leads to up-regulation of Hsp70 (a co-chaperone of Hsp90). The increase in Hsp70 chaperone subsequently inhibits the activity of MMP-2 and MMP-9, and thereby maintains the integrity of the extracellular matrix during wrinkle formation or skin aging. The other plausible reason for the low expression of MMP-2 and MMP-9 in 17AAG treated mice may be due to activation of some matrix degenerating enzymes activated through Hsp70 or UVR sensitive and insensitive signaling pathways. In contrast, it has been reported that chronic heat treatment in hairless mice causes skin wrinkle formation via oxidative damage (Shin, et al., 2012).

17AAG at a 500 nmol dose effectively inhibited SCC development. Since 17AAG had similar inhibitory activity when was applied post-UVR treatment, inhibitory effects of 17AAG cannot be attributable only to its sunscreen property. The mechanism by which topical 17AAG inhibits UVR-induced SCC is unclear. UVR-induced development of SCC accompanies expression and activation of several oncogenic signal transduction pathways (Hubbard et al., 2011). A few examples will be cited. UVR-induced downstream signaling components are mediated by the MAP kinase family including immediate early genes c-fos and c-jun, and transcription factors AP-1 and NF-κB (Huang et al., 1996). UVR also up-regulates Stat3 and NFAT transcription factors (Hubbard et al., 2011; (Zhang et al., 2001). The biological effects of UVR have been linked to the up-regulation of MAP kinases (ERKs, p38) (Sand et al., 2012). Further experimentation is needed to define the relative contribution of client proteins of Hsp90 in 17AAG-caused inhibition of UVR-induced development of SCC.

In sum, cutaneous SCC, whether developed in UVR exposed- or organ transplant populations, is a significant health problem (Lindelof et al., 2000; Ramsay et al., 2007; Bath-Hextall, et al., 2007). Although SCC can usually be cured by a variety of techniques, there are still an estimated 8,000 cases of nodal metastasis and 3,000 deaths in the United States annually. Hsp90, a molecular chaperone, plays a significant role in the stability and maturation of several oncogenic proteins (Goldman, 1998). The results presented (FIGS. 3-5) indicate that Hsp90 may be a potential molecular target for the prevention of UVR-induced development of cutaneous SCC. 17AAG can be formulated in cream for human use for prevention of SCC either developed in UVR exposed or organ transplant population.

Topical Application of Hsp90 Inhibitor 17-AAG to Mice Inhibits UVR-Induced Hsp90β-PKCε Interaction, Decreased PKCε Expression Levels and Inhibits UVR-Induced Development of SCC.

17-AAG is a competitive inhibitor of ATP binding and inhibits Hsp90 ATPase activity (9) Inhibition of Hsp90 ATPase activity by 17-AAG results in disruption of heteroprotein complexes and blocking of the refolding, conformational maturation and stability of transforming proteins including PKCε (2). To test whether disruption of UVR-induced interaction of Hsp90β with PKCε and other oncogenic client proteins by Hsp90 inhibitor 17-AAG prevents UVR-induced development of SCC, experiments were performed to determine whether 17-AAG treatment inhibits UVR-induced PKCε-Hsp90 interaction and development of SCC in mice. In these experiments, 17-AAG was applied topically to skin both before and after UVR exposure. Noteworthy observations are: 1) UVR exposure increased Hsp90β-PKCε interaction. 17-AAG treatment in conjunction with UVR exposure suppressed Hsp90β-PKCε interaction and inhibited PKCε protein level.

Initial tumor induction experiments were performed with SKH-1 hairless mice. Topical 17-AAG decreased SCC latency by 17 weeks and significantly (p=0.038) inhibited SCC incidence in SKH hairless mice. 17-AAG-caused inhibition of UVR-induced SCC accompanied inhibition of UVR-induced carcinomas and Hsp90β and PKCε expression levels. Interestingly, 17-AAG-caused inhibition of UVR-induced Hsp90β accompanied an increase in Hsp70 level. The effects of topical 17-AAG on UVR-induced development of SCC was further repeated in additional two strains of mice (wild-type and PKCε overexpressing transgenic FVB/N mice). The inventor has previously reported that epidermal PKCε level dictates the susceptibility of transgenic mice to the development of papilloma-independent SCC elicited by either the repeated exposure to UVR or using the DMBA-TPA tumor promotion protocol (30-38). As compared to wild-type littermates, PKCε over-expressing transgenic mice exhibit decreases in tumor latency and increases in SCC multiplicity on either FVB/N or SKH background (36). Again, topical application of Hsp90 inhibitor 17-AAG significantly (p<0.01) inhibited UVR-induced development of SCC.

Evaluation of Hsp90 Inhibitors' Effect on UV-Induced Hyperplasia

The inventor evaluated the effects of topically applied 17-AAG, 17-DMAG, NPV-BEP800, SNX-2112, AT-113387 and NVP-AUY922 on UVR-induced development of hyperplasia. Each of inhibitor was administered to two SKH1 mice. Treated mice were examined at 24 hr and 48 hr after they were exposed to UVR, respectively. As shown in Table 3, 17AAG and NPV-BEP800 had a positive effect on reducing the hyperplasia of both mice at both 24 hr and 48 hr post-UVR. NVP-AUY922 had a slightly negative effect on UVR-induced hyperplasia of both mice at 24 hr and 48 hr post-UVR. SNX-2112 had no effect on UVR-induced hyperplasia in mouse 1, however, it shows a positive effect on mouse 2 at both 24 hr and 48 hr post-UVR.

TABLE 3
Effect of Hsp90 inhibitors on UVR-induced hyperplasia

| | | Effect on Hyperplasia | | | |
|---|---|---|---|---|---|
| | | Mice 1 | Mice 2 | Mice 1 | Mice 2 |
| Serial | HSP90 | or Slide 1 | or Slide 2 | or Slide 1 | or Slide 2 |
| No. | Inhibitor | 24 Hr. post UVR | | 48 Hr. post UVR | |
| 1. | 17AAG | +++ | +++ | +++ | +++ |
| 3. | NPVBEP800 | +++ | +++ | +++ | +++ |
| 4. | SNX-2112 | −−− | +++ | −−− | +++ |
| 2. | 17DMAG | −−− | −−− | −−− | −−− |
| 5. | AT113387 | −−− | −− | −−− | −−− |
| 6. | NVPAU922 | −−− | −− | −− | −−− |

+++ (positive effect),
−−− (negative effect),
−− (less negative)

Other Hsp90 inhibitors of interest either have not been tested or have not been found to have a positive effect on reducing UVR-induced skin cancer at this time, but they may have potential for preventing UVR-induced skin cancer if a suitable administration method is identified.

### REFERENCES

### Each Incorporated Herein by Reference as if Set Forth in its Entirety

1. Anonymous. Skin Cancer; facts and figures. American Cancer Society, 2012.
2. Goldman G D: Squamous cell cancer: a practical approach. Semin Cutan Med Surg 1998, 17:80-95
3. Bangash H K, Colegio O R. Management of non-melanoma skin cancer in immunocompromised solid organ transplant recipients. Curr Treat Options, Oncol. 2012 September; 13(3):354-76.
4. Lindelöf B, Sigurgeirsson B, Gäbel H, Stern R S. Incidence of skin cancer in 5356 patients following organ transplantation. Br J Dermatol. 2000 September; 143(3):513-9.
5. Ramsay H M, Reece S M, Fryer A A, Smith A G, Harden P N. Seven-year prospective study of non-melanoma skin cancer incidence in U.K. renal transplant recipients. Transplantation. 2007 Aug. 15; 84(3):437-9.
6. Bath-Hextall F, Leonardi-Bee J, Somchand N, Webster A, Delitt J, Perkins W. Interventions for preventing non-melanoma skin cancers in high-risk groups. Cochrane Database Syst Rev. 2007 Oct. 17; (4):CD005414.
7. Bracher A, Hartl F U. Hsp90 structure: when two ends meet. Nat Struct Mol Biol. 2006; 13(6):478-80.
8. Wheeler D L, Martin K E, Ness K J, Li Y, Dreckschmidt N E, Wartman M, Ananthaswamy H N, Mitchell D L, Verma A K: Protein kinase C epsilon is an endogenous photosensitizer that enhances ultraviolet radiation-induced cutaneous damage and development of squamous cell carcinomas. Cancer Res 2004, 64:7756-7765.
9. Wheeler D L, Ness K J, Oberley T D, Verma A K: Protein kinase C epsilon is linked to 12-O-tetradecanoylphorbol-13-acetate-induced tumor necrosis factor-alpha ectodomain shedding and the development of metastatic squamous cell carcinoma in protein kinase C epsilon transgenic mice. Cancer Res 2003, 63:6547-6555.
10. Sand J M, Aziz M H, Dreckschmidt N E, Havighurst T C, Kim K, Oberley T D, Verma A K. PKC epsilon overexpression, irrespective of genetic background, sensitizes skin to UVR-induced development of squamous-cell carcinomas. J Invest Dermatol. 2010 January; 130(1):270-7.
11. Verma A K, Wheeler D L, Aziz M H, Manoharan H: Protein kinase C epsilon and development of squamous cell carcinoma, the nonmelanoma human skin cancer. Mol Carcinog 2006, 45:381-388.
12. Wheeler D L, Li Y, Verma A K: Protein kinase C epsilon signals ultraviolet light-induced cutaneous damage and development of squamous cell carcinoma possibly through Induction of specific cytokines in a paracrine mechanism. Photochem Photobiol 2005, 81:9-18.
13. Whitesell L, Santagata S, Lin N U. Inhibiting HSP90 to treat cancer: a strategy in evolution. Curr Mol Med. 2012 Jul. 17. [Epub ahead of print]
14. Grad I, Cederroth C R, Walicki J, Grey C, Barluenga S, Winssinger N, De Massy B, Nef S, Picard D. The molecular chaperone Hsp90α is required for meiotic progression of spermatocytes beyond pachytene in the mouse. PLoS One. 2010 Dec. 31; 5(12):e15770.
15. Taipale M, Krykbaeva I, Koeva M, Kayatekin C, Westover K D, Karras G I, Lindquist S. Quantitative analysis of hsp90-client interactions reveals principles of substrate recognition. Cell. 2012 Aug. 31; 150(5):987-1001.
16. Miyata Y, Nakamoto H, Neckers L. The Therapeutic Target Hsp90 and Cancer Hallmarks. Curr Pharm Des. 2012 Aug. 16. [Epub ahead of print].
17. Zuehlke A, Johnson J L. Hsp90 and co-chaperones twist the functions of diverse client proteins. Biopolymers. 2010 March; 93(3):211-7.
18. Sreedhar A S, Kalmár E, Csermely P, Shen Y F. Hsp90 isoforms: functions, expression and clinical importance. FEBS Lett. 2004 Mar. 26; 562(1-3):11-5.
19. Sõti C, Nagy E, Giricz Z, Vígh L, Csermely P, Ferdinandy P. Heat shock proteins as emerging therapeutic targets. Br J Pharmacol. 2005 November; 146(6):769-80.
20. Neckers L. Hsp90 inhibitors as novel cancer chemotherapeutic agents. Trends Mol Med. 2002; 8(4 Suppl):555-61.
21. Stebbins C E, Russo A A, Schneider C, Rosen N, Hartl F U, Pavletich N P. Crystal structure of an Hsp90-geldanamycin complex: targeting of a protein chaperone by an antitumor agent. Cell. 1997 Apr. 18; 89(2):239-50.
22. Neckers L. Chaperoning oncogenes: Hsp90 as a target of geldanamycin. Handb Exp Pharmacol. 2006; (172):259-77.
23. Biamonte M A, Van de Water R, Arndt J W, Scannevin R H, Perret D, Lee W C. Heat shock protein 90: inhibitors in clinical trials. J Med Chem. 2010. 14; 53(1):3-17.
24. Den R B, Lu B. Heat shock protein 90 inhibition: rationale and clinical potential. Ther Adv Med Oncol. 2012 July; 4(4):211-8.
25. Usmani S Z, Bona R, Li Z. 17 AAG for HSP90 inhibition in cancer—from bench to bedside. Curr Mol Med. 2009; 9(5):654-64.
26. Solit D B, Osman I, Polsky D, Panageas K S, Daud A, Goydos J S, Teitcher J, Wolchok J D, Germino F J, Krown S E, Coit D, Rosen N, Chapman P B. Phase II trial of 17-allylamino-17-demethoxygeldanamycin in patients with metastatic melanoma. Clin Cancer Res. 2008 Dec. 15; 14(24):8302-7.
27. Heath E I, Hillman D W, Vaishampayan U, Sheng S, Sarkar F, Harper F, Gaskins M, Pitot H C, Tan W, Ivy S P, Pili R, Carducci M A, Erlichman C, Liu G. A phase II trial of 17-allylamino-17-demethoxygeldanamycin in patients with hormone-refractory metastatic prostate cancer. Clin Cancer Res. 2008; 14(23):7940-6.
28. Gartner E M, Silverman P, Simon M, Flaherty L, Abrams J, Ivy P, Lorusso P M. A phase II study of 17-allylamino-17-demethoxygeldanamycin in metastatic or locally advanced, unresectable breast cancer. Breast Cancer Res Treat. 2012 February; 131(3):933-7. Epub 2011 Nov. 15.
29. Pacey S, Gore M, Chao D, Banerji U, Larkin J, Sarker S, Owen K, Asad Y, Raynaud F, Walton M, Judson I, Workman P, Eisen T. A Phase II trial of 17-allylamino, 17-demethoxygeldanamycin (17-AAG, Tanespimycin) in patients with metastatic melanoma. Invest New Drugs. 2012 February; 30(1):341-9. Epub 2010 Aug. 5.
30. Hubbard J, Erlichman C, Toft D O, Qin R, Stensgard B A, Felten S, Ten Eyck C, Batzel G, Ivy S P, Haluska P. Phase I study of 17-allylamino-17 demethoxygeldanamycin, gemcitabine and/or cisplatin in patients with refractory solid tumors. Invest New Drugs. 2011 June; 29(3):473-80. Epub 2010 Jan. 15.
31. Reagan-Shaw S., Nihal M., Ahmad N., Dose translation from animal to human studies revisited. The FASEB Journal. 2007 March; 22: 659-661.
32. U.S. Department of Health and Human Services, Food and Drug Administration, Center for Drug Evaluation and Research (CDER), Guidance for industry estimating the maximum safe starting dose in initial clinical trials for therapeutics in adult healthy volunteers. July, 2005.

We claim:

1. A method of preventing UV radiation-induced skin cancer, the method comprising the step of:
administering to a subject at risk of developing skin cancer an effective amount of an Hsp90 inhibitor,
wherein the skin cancer of the subject is prevented.

2. The method of claim 1, wherein the skin cancer is selected from the group consisting of basal cell carcinoma (BCC), squamous cell carcinoma (SCC) and malignant melanoma.

3. The method of claim 2, wherein the skin cancer is SCC.

4. The method of claim 1, wherein the Hsp90 inhibitor is selected from the group consisting of 17AAG, NVP-AUY922, Eleschlomol, Ganetespib, Alvespimycin, Geldanamycin, AT13387, KW-2478, SNX-2112, PF-04929113, NVP-BEP800, BIIB021, and a derivatives or analog thereof.

5. The method of claim 4, wherein the Hsp90 inhibitor is selected from the group consisting of 17AAG, NPV-BEP800 and SNX-2112 and a derivatives or analog thereof.

7. The method of claim 5, wherein the Hsp90 inhibitor is 17AAG.

8. The method of claim 5, wherein the Hsp90 inhibitor is NPV-BEP800.

9. The method of claim 1, wherein the subject is a human and the effective amount is a human equivalent dose converted from the effective amount for a non-human subject.

10. The method of claim 9, wherein the conversion of the human equivalent dose is based on Body Surface Area (BSA) factors.

11. The method of claim 1, wherein the administration is a topical administration.

12. A method of preventing UV radiation-induced skin damage comprising the step of administering an effective amount of at least one Hsp90 inhibitor to a subject at risk of UV radiation-induced skin damage,
wherein the skin damage is prevented.

13. The method of claim 12, wherein the skin damage is selected from the group consisting of wrinkle, loss of elasticity, aging of skin, age spots, freckles, dryness, uneven skin tone, and hyperplasia, or combinations thereof.

14. The method of claim 12, wherein the Hsp90 inhibitor is selected from the group consisting of 17AAG, NVP-AUY922, Eleschlomol, Ganetespib, Alvespimycin, Geldanamycin, AT13387, KW-2478, SNX-2112, PF-04929113, NVP-BEP800, BIIB021, and a derivatives or analog thereof.

15. The method of claim 14, wherein the Hsp90 inhibitor is selected from the group consisting of 17AAG, NPV-BEP800 and SNX-2112 and a derivative or analog thereof.

16. The method of claim 15, wherein the Hsp90 inhibitor is 17AAG.

17. The method of claim 15, wherein the Hsp90 inhibitor is NPV-BEP800.

18. The method of claim 12, wherein the subject is a human and the effective amount is a human equivalent dose converted from the effective amount for a non-human subject.

19. The method of claim 18, wherein the conversion of the human equivalent dose is based on Body Surface Area (BSA) factors.

20. The method of claim 12, wherein the administration is a topical administration.
